(19) United States
(12) Patent Application Publication
(10) Pub. No.: US 2005/0277842 A1
(43) Pub. Date: Dec. 15, 2005

(54) Monitoring respiratory movements device

(75) Inventor: Carlos Daniel Silva, Buenos Aires (AR)

Correspondence Address: NORRIS, MCLAUGHLIN & MARCUS, P.A., 875 THIRD AVE, 18TH FLOOR, NEW YORK, NY, 10022, US

(73) Assignee: Carlos Daniel Silva, Buenos Aires (AR)

(21) Appl. No.: 10/534,166

(22) PCT Filed: Nov. 6, 2003
(86) PCT No.: PCT/US03/35474

§ 371 Date: May 5, 2005

(30) Foreign Application Priority Data
Nov. 7, 2002 (AR) P020104280

(51) Int. Cl. A61B 5/08; A61B 5/103; A61B 5/117

(52) U.S. Cl. 600/534; 600/595

(57) ABSTRACT

The present invention relates to a monitor respiration movements device to be used on humans and also on animals for controlling the respiration movements and specially to control the apnea periods on infants. Furthermore the present invention is related to reduce the mortality rate caused by the sudden instant death syndrome (SIDS). The device comprises an accelerometer and a micro controller, said accelerometer includes a motion detector and a plurality of output plugs, said micro controller includes a plurality of input sockets, wherein said plurality of output plugs are connected so said plurality of input sockets and the micro controller includes signal outputs which are connected to an alarm means.

### BACKGROUND OF THE INVENTION

1. Field of the Invention

The present invention relates to a device for monitoring respiratory movements for controlling apnea periods both in humans and in animals. Furthermore the present invention is related to reduce the mortality rate caused by the sudden instant death syndrome (SIDS)

2. Description of the Prior Art

The study of respiratory movements and lung capacity is considered a major subject in the medicine filed. For such studies several methods and technologies have been used gathering every bit of information that could lead to a better detection and diagnosis of lung and respiratory dysfunction.

The use of spirometers dates since the 17^{th} century. The spirometers measure the lung capacity volume of a human being but they cannot measure the residual function capacity of the lungs. Another device used in such medical field is the pletismograph. The pletismograph allows achieving better and more complex studies of the abovementioned respiratory and lung disorders.

However, both the spirometers and the pletismographs results obtained by the use of such devices generated a limited result based on the data obtained by those devices.

Further, the use of transducers, the refurbishing of the signals obtained and the digital analysis of data, gave place to a better respiration monitoring by implementing the new technology to the common devices. Nevertheless there is still some situations where the respiration monitoring is not fully developed leading to several holes in that field. For example, while the removing from a patient the tubes from a life support machine, the patient is exposed to a tremendous risk where his body could not be prepared to breathe by it self. Since the moment the tubes of the life support machine are removed from the patient, there is no more monitoring of the patient so the doctors can not tell whether the patient is able to breath by him self not until a few vital seconds and even minutes had passed by, wherein some times those seconds or minutes could lead so death.

On the other hand, the Sudden Infant Death Syndrome (SIDS) is a medical disorder that claims the lives of many babies from one month to one year of age each year. Once known as crib death, these infant deaths remain unexplained after all known causes have been ruled out through autopsy, death scene investigation, and medical history. SIDS affects families of all races, religions, and income levels. It occurs during sleep, and strikes without warning. Its victims appear to be healthy. Neither parents nor doctors can tell which babies will die. The first year of life is a time of rapid growth and development when any baby may be vulnerable to SIDS.

The reason why SIDS happens to babies is still a mystery to find out, although researchers are making great progress in identifying deficits, behaviors, and other factors that may put an infant at higher risk. Scientists are exploring the development and function of the nervous system, the brain, the heart, and breathing and sleep patterns, body chemical balances, autopsy findings, and environmental factors. Researchers from several universities have, in fact, isolated a neurochemical defect in a portion of the brain of SIDS victims that controls the infant's protective responses to changes in oxygen and carbon dioxide levels. It appears likely that SIDS may be caused by some subtle developmental delay, an anatomical defect or functional failure. SIDS, like other medical disorders, may eventually have more than one explanation and more than one means of prevention. This may explain why the characteristics of SIDS babies seem so varied.

There are several technologies known in the art that monitors the respiration movements, some of them measure the pressure, some detects the electrical resistance variation taken from a transducer, while other technologies sense the respiration movements of the human body.

The problem for measuring the pressure values obtained by the respiration movements monitored from a human being or animal is to obtain reliable references to perform the tests. To overcome the mentioned problem there are two major technologies used to monitor the apnea in babies. The first one uses a pressure transducer, which is placed under the mattress to monitor the baby's respiration movements. The second technology consists in adhesively attaching a balloon on the baby's abdomen, connecting said balloon to a pressure transducer. The variations in the electrical resistance must be detected by the use of a belt placed around the baby's body.

When using a transducer under the mattress, as mentioned before, the changes of pressure produced by the respiration movements are partially absorbed by the mattress itself therefore the reading obtained by the transducer as not quite accurate. In the event that a balloon is attached to the baby's abdomen, the reading a rally accurate compared to the technology described above, but since the balloon has to be attached to the baby's abdomen by an adhesive material, said adhesive material prevents from using the balloon in babies for more than 8 month, since skin reactions may appear leading into a rash and making the baby very uncomfortable. Furthermore, while monitoring the baby's respiration movements one must avoid the use of wiring in such devices since no only the baby tends to play with the wiring and could lead to a malfunction of the equipment but also it could represent a big danger to him due to risk of choking.

Additionally, the monitoring in animals is still under major development since there are no new methods or technologies applied in this field. The monitoring of the respiration movements in animals such as in stallions, and the like has increased significantly. Nowadays there are several veterinarian therapies to be performed on animals, being those therapies very similar to the ones used on humans, including the use of life support devices. However the monitoring techniques are still very limited. One of the major problems involved in the monitoring techniques is the difficulty of placing the wiring, catheter, sensors and the like in the animal.

To overcome the drawbacks aforementioned there is a need for a respiration movement monitor that can be easily handled, compact, reliable in the reception of signals, and that will not interfere with the respiration movements of the human or animal.

### SUMMARY OF THE INVENTION

It is therefore one object of the present invention to provide a monitoring respiratory movements device to be used both in humans and animals for controlling the respiration movements and specially the apnea periods in infants to reduce the mortality rate caused by the sudden instant death syndrome (SIDS).

It is still another object of the present invention to provide a monitoring respiratory movement device for improving the monitoring techniques and methods used in the veterinary filed for monitoring stallions and the like.

It also another object of the present invention to provide a monitoring respiratory movement device that uses an accelerometer as a sensor, since there are acceleration motion in the respiration movements that can be monitored. This accelerometer is placed in a silica chip using nanotechnology resulting in a device of really low mass and with enough sensibility to detect the acceleration motion in the respiration movements. By use of a micro controller and with software associated, the device can perform every necessary function to fashion the signals received from the accelerometer, transmit them and eventually setting off and alarm.

It is a further object of the present invention to provide a monitor respiratory movement device to be used on both humans and animals for controlling the respiratory movements wherein the devices comprises an accelerometer, a micro controller, said accelerometer includes a motion detector and a plurality of output plugs, said micro controller includes a plurality of input sockets; wherein said plurality of output plugs are connected so said plurality of input sockets and the micro controller includes signal outputs which are connected to an alarm means.

The above and other objects, features and advantages of this invention will be better understood when taken in connection with the accompanying drawings and description.

### BRIEF DESCRIPTION OF THE DRAWINGS

The present invention is illustrated by way of example in the following drawings wherein:

FIG. 1 shows a block diagram of the electronic circuit of the device of the present invention;

FIG. 2 shows the electronic circuit of the device of the present invention;

FIG. 3 is an embodiment of the electronic circuit of the device of the present invention;

FIG. 4 is a perspective view taken from the back of a holder for the device of the present invention.

### DESCRIPTION OF THE PREFERRED EMBODIMENTS

As stated before, even though the present invention can be used either for human o animal respiration monitoring, the following description is based exclusively in the monitoring of respiration movements of infants, and specially in one month to one year old babies. Therefore, the following example should not be considered as a limit to the scope and spirit of the present invention.

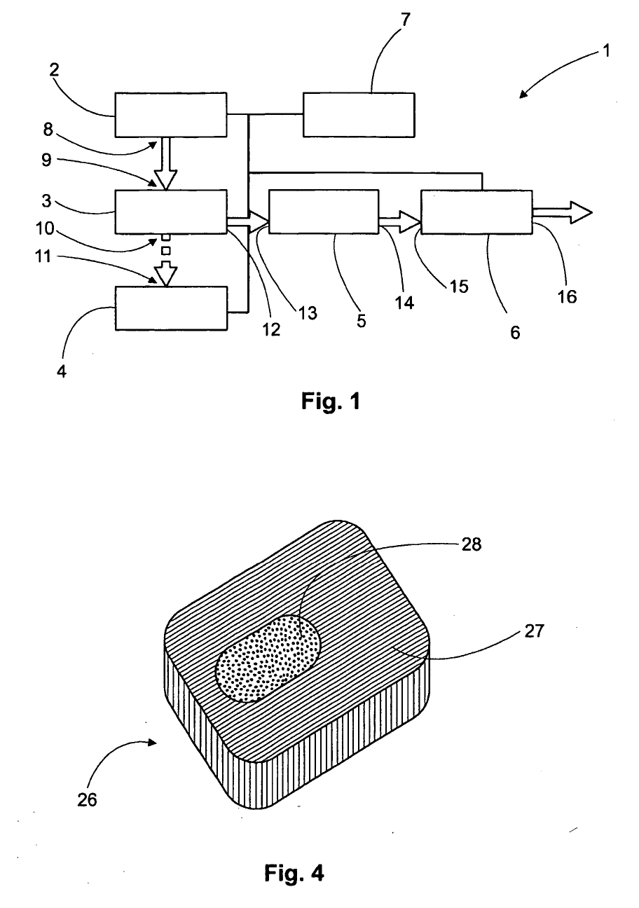

Now referring in detail to FIG. 1, the monitoring respiration movement device is defined by an electronic circuit generally described with the reference number 1. Said electronic circuit 1 comprises an accelerometer 2 including a motion detector, such as the one showed in FIG. 4. A micro controller 3, alarm means 4, instant acceleration transmission means defined by a series/parallel converting module 5 and a signal transmission module 6, and a feeding source 7 are also connected to the electronic circuit 1.

The accelerometer 2, being in this embodiment an ADXL202, is an accelerometer of a very high sensitivity and a very low mass. These characteristics allow to not interfering with the normal respiratory movements of the infant. The accelerometer 2 includes digital signal outputs 8 connected to respective inputs 9 of the micro controller 3. The micro controller 3 includes an output 10 from where the alert signals are sent to the input 11 of the alarm means 4.

The micro controller, being in this embodiment a PIC16F87 model, has implemented every necessary function to read the signals of the output 8 of the accelerometer 2. According to the software used in the micro controller 3, several signals from output 12 can be sent to the inputs 13 of the series/parallel converting module 5 and then from the outputs 14 to the inputs 15 of the transmission module 6, to send from the outputs 16 the signals from the accelerometer 2 towards others signal processing devices. Said transmission of signals can be either galvanic or wireless depending on the transmitting module to be used.

It can be uses as a processing device a computer where the processing of the signals will be subject to the software used in the same. If the processing it taken in a laboratory with animals, a galvanic transmission can be safely used. The data should be input trough one of the serial ports (RS232) in the computer. For a wireless transmission of the signals an electromagnetic signal can be easily used, being the most common used signals the radio frequency signals and the infrared signals. However the device of the present invention can be equipped with ultrasound equipment, being these methods of transmitting the signals obtained from the accelerometer 2 not to be considered as limiting the scope of the present invention.

The converting module 5 included in the micro controller 3, sends the signals to the transmission module 6. In this embodiment the transmission module 6 comprises two integrated circuits defining an encoder such as a MCP2150 which encodes the received signal in such a fashion that can be transmitted by an infrared transmitter 6′ (e.g. TFDS4500). The infrared transmitter 6′ is an IrDA certified transmitter which transmits data at the speed of 115.2 Kb/sec.

By means of the converting module 5 the parallel n bits signals delivered by the micro controller 3 are converted to a series of n bits, which are added to perform the necessary control tasks. In this embodiment the accelerometer 2 has a 12 bits resolution, however only the more significant 8 bits were used in the assays.

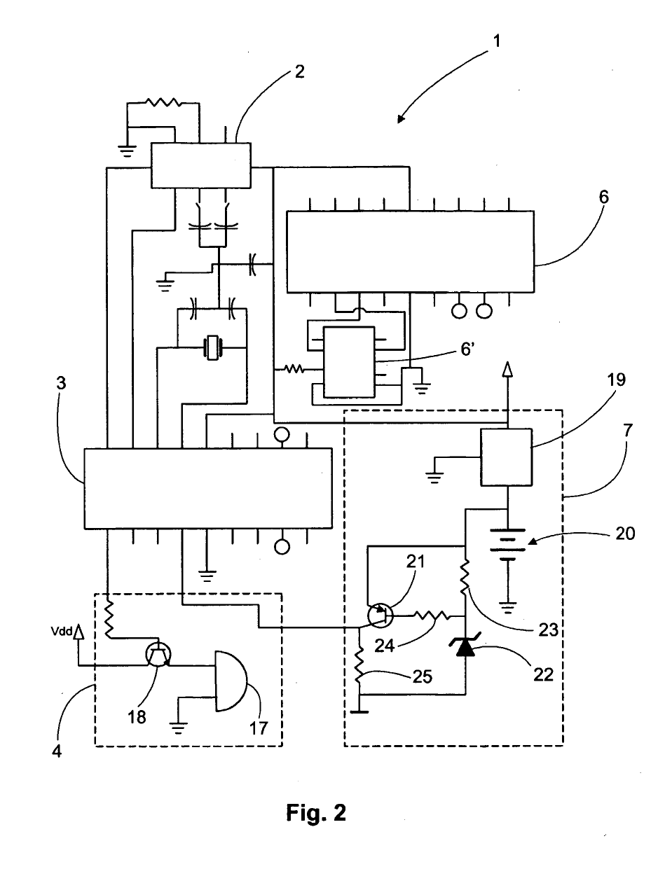

Referring now to FIG. 2 the alarm means 6 comprises a buzzer 17 connected to output 10 of the micro controller 3 through a transistor 18. The alarm means 6 can present several settings. For example, based on a multivibe circuit and a speaker attached to it or the speaker can be replaced by a LED or even a combination of both. Still referring to FIG. 2, the power supply 7 is defined by a voltage regulator 19 such as a 78L05 voltage regulator. The voltage regulator 19 is connected to the electric network through a transformer and a battery 20 associated to voltage regulator circuit configured based on a transistor 21 and a Zener diode 22 with their corresponding polarization resistors 23 to 25. In the event that the device of the present invention is used in humans, the power supply 7 delivers DC power required for the proper performance of the circuit from the battery 20. By doing so, the device does not need to be connected to the electric network, protecting the integrity of the human being.

The accelerometer 2 sends modulated signals to the micro controller 2 by means of the DMC corresponding to the instant acceleration measured in two orthogonal axes. The micro controller 2 includes software that demodulates the received signals sent in series to the transmitter module 6. The transmitter module 6 comprises the IrDA decoder and an IR transducer. In the event that the device of the present invention should be used as only an apnea monitor, the software detects the variations in the acceleration detected by the accelerator 2. If no variations are detected in a period of T=20 seconds, the micro controller's software will set off the alarm means 4.

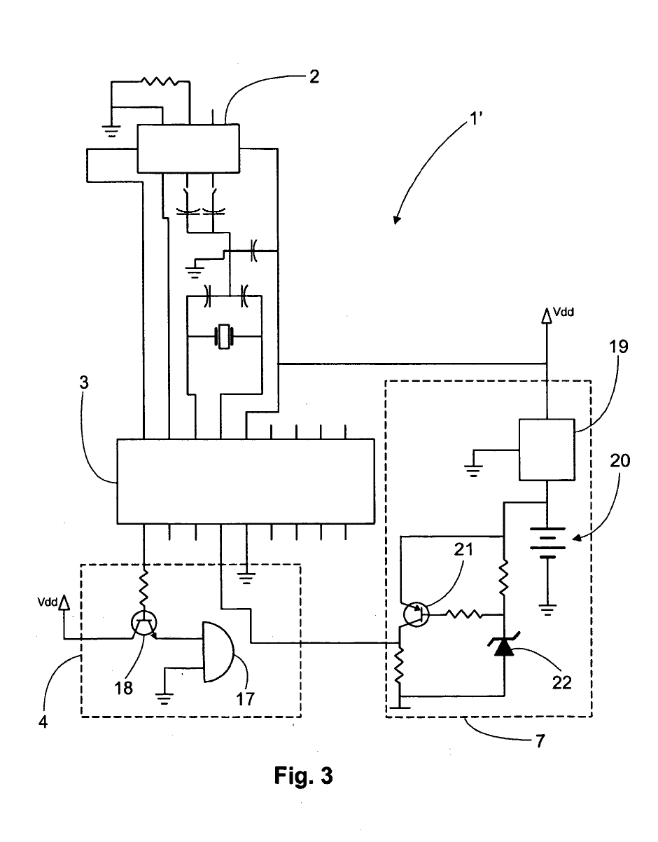

Depending on the use of the device of the present invention (e.g. as an apnea monitor for preventing SIDS), only the alarm means 4 should be connected to the micro controller 3, avoiding the use of the converting module 5 and the transmission module 6. This embodiment of the device is illustrated in FIG. 3, wherein the electronic circuit of the device is identified by the reference number 1′. Accordingly, the performance of the device using the electronic circuit 1′ is exactly the same as the performance of the device using the electronic circuit 1, except for the absence of the converting module 5 and the transmission module 6.

Referring now to FIG. 4 it is shown the geometrical configuration of the device of the present application, specially the shape of the holder 26 inside of which is hosted either electronic circuits 1 or 1′. Further, it can be seen the motion sensors 27 places in a back end wall 26′ of the holder 26. For proper functioning the device must be kept in a stable and steady position with respect to the body of either the human or animal. Preferably, the device should be places in the trunk zone of the body where the respiration movements are more easily detected. Since the device of the present invention does not need to be in touch with the skin of the human, the device can be wore over the subject's garment.

The invention in its broader aspects is not limited to the specific details shown and described above. Departures may be made from such details within the scope of the accompanying claims without departing from the principles of the invention and without sacrificing its advantages.

1. A monitor respiratory movement device to be used on both humans and animals for controlling the respiratory movements wherein the monitor comprises:
a) an accelerometer
b) a micro controller
c) said accelerometer includes a motion detector and a plurality of output plugs
d) said micro controller includes a plurality of input sockets; wherein said plurality of output plugs are connected so said plurality of input sockets and the micro
controller includes signal outputs which are connected to an alarm means.

2. The device of claim 1 wherein said micro controller comprises outputs connected to transmission means for transmitting the signals sent by the accelerometer towards external processing devices for processing said signals.

3. The device of claim 2 wherein said external processing devices are computer means.

4. The device of claim 2 wherein said transmission means are instant acceleration transmission modules.

5. The device of claim 4 wherein said instant acceleration transmission modules comprises:
a) a series/parallel signal converter module;
b) a transmission module;
wherein said series/parallel signal converter module is an integral part of the micro controller and the output sockets of the same are connected to said transmission module; being that transmission module defined by a signal codification integrated circuit and a signal transmission integrated circuit.

6. The device of claim 5 wherein said signal transmission integrated circuit is an infrared transmission device.

7. The device of claim 5 wherein said signal codification integrated circuit is an IrDA signal encoder certified.

8. The device of claim 1 wherein said alarm means is a buzzer connected to the micro controller through a transistor.

9. The device of claim 1 where in current supply for the device is provided by an integrated regulating tension circuit connected to the electric network through a transformer and a CC battery associated to a regulating tension circuit configured based on a transistor and a Zener diode.
